(19) United States
(12) Patent Application Publication
(10) Pub. No.: US 2020/0246411 A1
(43) Pub. Date: Aug. 6, 2020

(54) ANTI-DIARRHOEAL HERBAL COMPOSITIONS

(71) Applicants: Adithya SAGAR, Gainesville, FL (US); Anusree Suseela SASIDHARAN, Trivandrum (IN); Paolo GEORGE, Cochi (IN); SRH NUTRITION LLP, Trivandrum (IN); SYMEGA FLAVOURS INDIA PVT. LTD., Cochin (IN)

(72) Inventors: Adithya SAGAR, Gainsville, FL (US); Anusree Suseela SASIDHARAN, Trivandrum (IN); Paolo GEORGE, Kochi (IN); Ragitha Vijaya MURUKAN, Trivandrum (IN); Swathy GIRIDHARAN, Kollam (IN)

(21) Appl. No.: 16/652,186

(22) PCT Filed: Sep. 29, 2018
(86) PCT No.: PCT/IN2018/050623

§ 371 Date: Mar. 30, 2020

(30) Foreign Application Priority Data
Sep. 29, 2017 (IN) 201721027016

(51) Int. Cl. A61K 36/53; A61K 36/75; A61K 36/23; A61K 36/54; A61P 1/12

(52) U.S. Cl. CPC A61K 36/53; A61K 36/75; A61P 1/12; A61K 36/54; A61K 36/23

(57) ABSTRACT

The present synergistic herbal compositions comprising herbal ingredients extracted from at least two herbal species selected from the group consisting of Ocimum basilicum, Agathosma betulina, Cinnamomum cassia, Cinnamomum verum and Apium graveolens helps maintain the electrolyte and fluid balance by exerting its inhibitory secretory activity and by increasing the absorptive capacity of the intestine to prevent dehydration during an episode of infectious and non-infectious diarrhoea and in dehydration associated with various clinical and non-clinical conditions

### TECHNICAL FIELD OF THE INVENTION

The present invention relates to synergistic herbal compositions having anti-diarrhoeal properties. More particularly, the present synergistic herbal compositions help maintain the electrolyte and fluid balance by exerting its inhibitory secretory activity and by increasing the absorptive capacity of the intestine to prevent dehydration during an episode of diarrhoea and diarrhoea related diseases.

### BACKGROUND AND PRIOR ART OF THE INVENTION

Diarrhoeal disease is a leading cause of child mortality and morbidity in the world, and mostly results from contaminated food and water sources. Even though diarrhoea is a treatable, preventable and curable disease, about 1.7 billion cases of childhood diarrhoeal disease are reported worldwide per year. Diarrhoea due to infection is widespread throughout developing countries. In low-income countries, children under three years old experience on an average three episodes of diarrhoea every year. Diarrhoea is the third most common cause of death in under-five children in India, responsible for 13% deaths in this age-group, killing an estimated 300,000 children in India each year (S. Lakshminarayanan et al, J Nat Sci Biol Med. 2015 January-June; 6(1): 24-28).

Each episode of diarrhoea deprives the child of the nutrition necessary for growth. The repeated occurrence of diarrhoea is considered as the single most important cause of malnutrition, therefore the global incidence of diarrhoea parallels with global incidence of malnutrition. Malnourished patients, especially children, have little resistance to any infection and dehydration secondary to diarrhoea often causes death. During a diarrhoeal episode, water and electrolytes including sodium, chloride, potassium and bicarbonate are lost through liquid stools, vomit, sweat, urine and breathing. Dehydration occurs when these losses are not replaced adequately and a deficit of water and electrolytes develops resulting in metabolic acidosis (http://www.who.int/mediacentre/factsheets/fs330/en/).

The different phenomena occurring in the gastrointestinal system in a diarrhoeal disease condition is discussed herein below.

Leaky Gut Syndrome:

Campylobacter, Salmonellae, Clostridium difficile and Shigella are the most common causes of bacterial diarrhoea. Less common causes are Escherichia coli, Yersinia and Listeria. Rotavirus is the most common cause of diarrhoeal disease among infants and young children. Repeated episodes of diarrhoea caused due to bacterial infection destroy the mucosal wall of the intestine, thereby causing multiplying of bacteria inside the cells, resulting in Leaky gut syndrome. Antigenic molecules leak from the gut into the blood thus reacting with antibodies causing local and systemic inflammation in gastrointestinal tract. Additionally, villus damage occurs and further absorption of electrolytes, nutrients, vitamins, amino acids are not possible resulting in malnutrition. All these factors will lead to a clinical condition known as environmental enteropathy. India is in an environmental enteropathy belt.

During diarrhoea, routine fluid absorption by the intestine does not occur resulting in a loss of massive amount of fluid. Loosening of stools occurs when daily faecal water output increases by 50-60 mL. An increase in faecal water excretion of 100 mL is sufficient to increase stool weight above 200 g/24 h, i.e. the upper limit of normal. Thus, a decrease in the overall intestinal water absorption of only 1% to 2% is sufficient to cause diarrhoea.

Intestinal Electrolyte Transport:

The small intestine is the portal for absorption serving to absorb water and essentially all dietary organic molecules to the body. The small intestine also plays a critical role in water and acid-base balance. It has millions of small projections called villi which extend about 1 mm into the lumen. Villus is covered predominantly with mature, absorptive cell called enterocytes, along with occasional mucus-secreting goblet cells. These cells survive only for a few days and are shed into the lumen to become part of the ingesta to be digested and absorbed. Villi increase the surface area for absorption. Around the villi are moat-like invaginations of the epithelium called crypts (of Lieberkuhn). Crypts are lined largely with younger epithelial cells which are involved primarily in secretion. Towards the base of crypts are stem cells. These stem cells continually divide and provide the source of all the epithelial cells in the crypts and on the villi. One daughter cell from each stem cell division is retained as a stem cell. Daughter cells mature and differentiate as they migrate from the crypt into the villi. The cells are fully differentiated by the time it reaches the tip of the villi.

With maturation and differentiation, the epithelial cells acquire new transport proteins that are essential for absorption of electrolytes, nutrients and other minerals, so that a fully matured villus epithelial cell has all necessary transport machineries like SGLT1; for sodium coupled glucose transport, NHE3; for proton-sodium exchange, amino acid transporters, vitamin transporters and mineral transporters. Expression of disaccharidases also occurs on the tip of the villi where matured and differentiated epithelial cells occur. Disaccharidases are essential for breaking down disaccharides into monosacharides during the digestion of carbohydrates. Digestion of carbohydrates starts in the mouth by salivary amylase and is broken down into disaccharides by pancreatic amylase. Conversion of disaccharides into monosaccharides occurs by activity of disaccharidases located at the tip of the villi, where matured and differentiated epithelial cells are located. The other becomes committed to differentiate along one of four pathways to become an enterocyte, an enteroendocrine cell, a goblet cell or a Paneth cell. Cells in enterocyte lineage divide several more times as they migrate up the crypts, and as they migrate onto the villi, they differentiate further into mature absorptive cells (enterocytes) that express more number of transport proteins and enzymes. Absorption in villus is driven by sodium and chloride ions.

In the intestine, electrogenic absorption of sodium and chloride occur. Sodium is absorbed through Na²⁺/H⁺ exchanger (NHE2/NHE3). Chloride is absorbed through Cl⁻/HCO₃⁻ exchanger (anionic exchanger). When sodium and chloride are absorbed, along with it water is also absorbed. Another transporter which absorbs sodium is the Sodium Coupled Glucose Transporter (SGLT), an electrogenic absorption in which for one glucose molecule two sodium ions are absorbed. In the crypt region of the intestine secretion occurs through anionic transporters mainly Cystic Fibrosis Transmembrane Conductance receptor (CFTR).

In a normal absorptive small intestine, there is a fine balance between absorption occurring in the villus cell region and the secretion from the crypt cells. An imbalance resulting from a decreased absorption, increased secretion, or a combined effect can result in diarrhea.

When diarrhoea occurs all these absorptive mechanisms are disturbed. Thus, diarrhoea occurs due to (a) an increased secretion mediated by secretagogues like cAMP, Ca²⁺, cGMP; (b) decreased absorption as a result of structural damage to the villi causing reduced villus height; (c) decreased number of transporters; (d) inhibition of transporters, or (e) by a combination of increased secretion and decreased absorption.

To control diarrhoea, sufficient hydration of the patient is required by providing the necessary ions to maintain electrolyte balance. The treatment of choice is administration of oral rehydration solution (ORS), which has reduced the levels of mortality in children and elderly (6-7 million) but not morbidity.

The rate of rehydration in diarrheal patients is not in step with the rate of electrolyte loss. Maintenance of hydration is a critical element in the treatment of diarrheal diseases. Currently, secretory diarrhea is treated with an oral rehydration drink (ORD) a salt solution containing sodium and a significant amount of glucose and other sugar molecules. Glucose has always been a mainstay in both enteral and parenteral fluids for correcting electrolyte and nutrient absorption defects associated with disease conditions. ORDs are designed to correct the loss of fluids and electrolytes in secretory diarrhea.

The treatment is based on active absorption of glucose by sodium coupled glucose transporter, during the intestinal infection lend to the co-transport of Na⁺ ions and water absorption. ORS is useful to treat dehydration caused by diarrhoea, but it does not decrease the stool volume and stool frequency. Further, ORS cannot be administered to patients with diabetes and severe vomiting. Depending on the severity of diarrhoea, in some cases ORS is not enough, resulting in usage of antibiotic, spasmolytic, and antiprotozoal drugs.

The disadvantages and side-effects encountered on administration of conventionally used drugs in the treatment of diarrhoea are detailed in the table herein below.

| Drug | Disadvantages |
|---|---|
| Codeine | Produces secondary effects such as nausea, dizziness and acts against |
| | the central nervous system. Continuous use can induce physical |
| | dependence and addiction. |
| Loperamide | Frequent adverse reactions induced are hypersensibility reactions |
| | (cutaneous eruption), gastrointestinal disorders (constipation, colic, |
| | abdominal distention, nauseas and vomit), fever and dry mouth. It is a |
| | non-prescription drug for children since it causes CNS depression. |
| Diphenoxylate | Therapeutic doses induce adverse reactions in the CNS (confusion, |
| | sedation, depletion, cephalea), allergic reactions (anaphylaxis, pruritus) |
| | on gastrointestinal apparatus (toxic megacolon, paralytic ileum, vomit, nauseas |
| | and abdominal pain) It can cause euphoria and has an analgesic |
| | effect. Diphenoxylate is contraindicated in children younger than 2 years old. |
| Bismuth | Causes adverse reactions (dizziness, cephalea, constipation, dark stools, |
| | ataxia, tremor, encephalopathy, confusion, delirium and convulsions). |
| Racecadotril | Cause some adverse reactions such as hypokalemia, bronchospasm, |
| | fever, vomit and otitis. |
| Clonidine | Causes an alteration of gut motility with effect on intestinal transport, it |
| | causes hypotension. |

Approximately, 80% of the world's population uses medicinal plants to treat health problems. Patent literature as well as research articles have emphasized the use of herbal ingredients in the cure and prevention of diarrhoea.

PCT Publication WO2011083398 discloses an herbal composition for diarrhoea comprising blend of extract of Aegle marmelos, Cyperus rotundus, Holarrhena antidysenterica, Mangifera indica and Zingiber officinale and a pharmaceutically acceptable carrier. However, the invention disclosed therein only demonstrates the delay in onset of diarrhoea on inducing diarrhoea by castor oil administration.

Further, Indian Patent No. 245725 and U.S. Pat. No. 6,039,954 disclose herbal compositions for the treatment of gastrointestinal diseases, but fail to address the disruption of the villi, crypt cells, electrolyte/fluid imbalance caused due to diarrhoea or due to bacterial infections.

Prior art literature has not evidenced a synergistic composition that combats the disruptive effects caused by a diarrhoea episode ensuring both rehydration and electrolyte absorption.

In order to provide an efficient composition that is safe and does not have any side effects, the present inventors have screened herbal extracts/oils for their anti-diarrhoeal activity and have devised anti-diarrhoeal compositions which repair/maintain the structural integrity of the villi of the intestine, thereby achieving (a) anti-secretory activity against secretagogues such as cAMP, Ca²⁺, cGMP which cause diarrhoea, and (b) increasing absorption capacity of sodium and chloride in the intestine.

### OBJECT OF THE INVENTION

It is an object of the present invention to provide a synergistic herbal composition possessing anti-diarrhoeal properties including;
(i) increasing the non-nutrient and nutrient dependent electrolyte absorption from matured and differentiated villus epithelial cells of the intestine by decreasing active chloride secretion from the crypt of the intestine; and by increasing absorption from the crypt;
(ii) decreasing the concentration of intracellular secretagogues such as cAMP, cGMP, and Ca²⁺;
(iii) directly inhibiting anion channels, or decreasing the expression of secretory anion channels from the membrane or by increasing the turn-over rate of channels on the brush border membrane for absorption;
(iv) increasing electrolyte absorption by increasing the cell turn-over rate (increased mitotic division in the crypt of the small intestine);
(v) increasing electrolyte absorption by increasing the height of villus (increased mitotic activity of crypt stem cells).

### SUMMARY OF THE INVENTION

In an aspect, the present invention provides a synergistic herbal composition having anti-diarrhoeal properties, comprising herbal ingredients extracted from at least two herbal species selected from the group consisting of Ocimum basilicum (basil), Agathosma betulina, Cinnamomum cassia (cassia), Cinnamomum verum (bark of Cinnamomum) and Apium graveolens (celery). The present invention provides an herbal composition comprising at least two herbal ingredients selected from the group consisting of Basil oleoresin, Buchu leaf oil, Basil oil, Cassia oil, Cinnamon bark oil and Celery seed oil.

In another aspect, the present invention provides the herbal composition to further comprise one or more electrolytes selected from the group comprising sodium (Na⁺), Chloride (Cl⁻), Bicarbonate (HCO₃⁻), Potassium (K⁺), Phosphate anions (H₂PO₄, HPO₄²⁻), Magnesium (Mg²⁺), Calcium (Ca²⁺) and sulphate (SO₄²⁻).

In yet another aspect, the present invention provides a synergistic herbal composition comprising active ingredients selected from the group consisting of oleoresin extracted from Ocimum basilicum, oil extracted from leaves of Agathosma betulina, oil extracted from Cinnamomum cassia, oil extracted from bark of Cinnamomum verum and oil extracted from seeds of Apium graveolens and a pharmaceutically acceptable carrier.

The herbal compositions provided in the present invention possess (a) anti-secretory activity against secretagogues like cAMP, Ca²⁺, cGMP which cause diarrhoea, and (b) increases absorption capacity of electrolytes from the intestine

The present invention provides a method of treating diarrhoea, diarrhoea related disorders and electrolyte and fluid imbalance, the said method comprising administering an herbal composition comprising at least two herbal ingredients selected from the group consisting of Basil oleoresin, Buchu leaf oil, Basil oil, Cassia oil, Cinnamon bark oil and Celery seed oil and one or more electrolytes to an individual exhibiting symptom of diarrhoea.

The present invention provides use of herbal compositions comprising active ingredients selected from the group consisting of oleoresin extracted from Ocimum basilicum, oil extracted from leaves of Agathosma betulina, oil extracted from Cinnamomum cassia, oil extracted from bark of Cinnamomum verum and oil extracted from seeds of Apium graveolens and a pharmaceutically acceptable carrier, for treating diarrhoea, diarrhoea related disorders and electrolyte and fluid imbalance.

### BRIEF DESCRIPTION OF DRAWINGS

FIG. 1 depicts the effect of (A) Buchu leaf oil, (B) Basil oil, (C) Cassia oil, (D) Cinnamon bark oil, (E) Celery seed oil and (F) Basil oleoresin on inherent anion secretion. The aforesaid herbal extracts decreased anion secretion (delta I_{sc}) at 30 minutes, 45 minutes and 1 hour;

FIG. 2 depicts the effect of (A) Buchu leaf oil and (B) Basil oil on forskolin (cAMP) induced anion secretion. The aforesaid herbal extracts significantly decreased forskolin induced chloride secretion (delta I_{sc});

FIG. 3 depicts the effect of (A) Buchu leaf oil, (B) combination of buchu leaf oil and cassia oil, (C) cinnamon bark oil and celery seed oil and a (D) combination of buchu leaf oil, basil oleoresin, cassia oil, cinnamon bark oil and celery seed oil on forskolin induced anion secretion;

FIG. 4(a) depicts Western blot analysis showing the effect of basil oil on protein levels of CFTR (a cAMP-activated chloride channel) and SGLT1 (sodium-coupled glucose transporter 1) in the brush border membrane of small intestinal tissues from mice, both in the presence and absence of forskolin;

FIG. 4(b) depicts Western blot analysis showing the effect of cinnamon bark oil (CBO) on the protein levels of CFTR and SGLT1 in the brush border membrane isolated from mouse small intestine, both in the presence and absence of forskolin;

FIG. 4(c) depicts Western blot analysis showing the effect of the formulation on the protein levels of CFTR and SGLT1 during secretagogue induced diarrheal conditions (formulation comprises a combination of Basil oil, Cinnamon bark oil, Cassia oil and Buchu leaf oil);

FIG. 5(a) depicts the effect of basil oil (BO) on fluid absorption using in vitro loop assay; FIG. 5(b) depicts the effect of cinnamon bark oil (CBO) on net fluid absorption using in vitro loop assay to study its effect on basal and forskolin-stimulated conditions; FIG. 5(c) depicts the Effect of cassia oil (CO) on net fluid absorption in mouse small intestine: in vitro loop assay; FIG. 5(d) depicts the effect of buchu leaf oil (BLO) on fluid absorption in mouse small intestine using in vitro loop assay.

FIG. 6 depicts a representative saturation kinetic curve showing the method to arrive at the concentration of buchu leaf oil to inhibit specific channels responsible for electrolyte loss and therefore effective in increasing net fluid absorption

### ABBREVIATIONS USED

Isc: short circuit current

Km: The Michaelis constant

Vmax: Maximum velocity

### DETAILED DESCRIPTION OF THE INVENTION

Geographical Source of Biological Material Used in the Present Invention:

Basil oleoresin extracted from Ocimum basilicum is procured from India,

Buchu leaf oil extracted from Agathosma betulina is procured from South Africa,

Basil oil extracted from Ocimum basilicum is procured from India,

Cassia oil extracted from Cinnamomum cassia is procured from Indonesia,

Cinnamon bark oil extracted from Cinnamomum verum is procured from Sri Lanka, and Celery seed oil extracted from Apium graveolens is procured from Egypt.

The invention will now be described in detail in connection with certain preferred and optional embodiments, so that various aspects thereof may be more fully understood and appreciated.

In a preferred embodiment, the present invention provides an herbal composition comprising at least two herbal ingredients selected from the group consisting of Ocimum basilicum, Agathosma betulina, Cinnamomum cassia, Cinnamomum verum and Apium graveolens and a pharmaceutically acceptable carrier.

Accordingly, the herbal composition comprises at least one component selected from an oleoresin extracted from Ocimum basilicum, and essential oils selected from the group comprising buchu leaf oil is extracted from Agathosma betulina, Basil oil is extracted from Ocimum basilicum; Cinnamomum cassia oil, cassia oil from the bark of Cinnamomum verum and celery oil from the seed of Apium graveolens.

The present synergistic herbal composition comprises herbal ingredients in a concentration ranging from 50 ml/Kg to 300 ml/Kg.

The following are the embodiments that the present invention provides with regards to the herbal compositions employed herein.

In an embodiment, the present invention provides a synergistic herbal composition, wherein the said composition comprises;
(a) basil oil in a concentration ranging from 0.027 μL/kg to 57 μL/kg; and
(b) cinnamon bark oil in a concentration ranging from 0.33 μL/kg to 22 μL/kg.

In an embodiment, the present invention provides a synergistic herbal composition, wherein the said composition comprises;
(a) basil oil in a concentration ranging from 0.027 μL/kg to 57 μL/kg; and
(b) cassia oil in a concentration ranging from 0.67 μL/kg to 50 μL/kg.

In an embodiment, the present invention provides a synergistic herbal composition, wherein the said composition comprises;
(a) basil oil in a concentration ranging from 0.027 μL/kg to 57 μL/kg; and
(b) buchu leaf oil in a concentration ranging from 0.33 μL/kg to 25 μL/kg.

In an embodiment, the present invention provides a synergistic herbal composition, wherein the said composition comprises;
(a) basil oil in a concentration ranging from 0.027 μL/kg to 57 μL/kg; and
(b) celery oil in a concentration ranging from 0.0008 μL/kg to 8 μL/kg.

In an embodiment, the present invention provides a synergistic herbal composition, wherein the said composition comprises;
(a) basil oil in a concentration ranging from 0.027 μL/kg to 57 μL/kg; and
(b) basil oleoresin in a concentration ranging from 0.002 μL/kg to 23 μL/kg.

In an embodiment, the present invention provides a synergistic herbal composition, wherein the said composition comprises;
(a) cinnamon bark oil in a concentration ranging from 0.33 μL/kg to 22 μL/kg; and
(b) cassia oil in a concentration ranging from 0.67 μL/kg to 50 μL/kg.

In an embodiment, the present invention provides a synergistic herbal composition, wherein the said composition comprises;
(a) cinnamon bark oil in a concentration ranging from 0.33 μL/kg to 22 μL/kg; and
(b) buchu leaf oil in a concentration ranging from 0.33 μL/kg to 25 μL/kg

In an embodiment, the present invention provides a synergistic herbal composition, wherein the said composition comprises;
(a) cinnamon bark oil in a concentration ranging from 0.33 μL/kg to 22 μL/kg; and
(b) celery seed oil in a concentration ranging from 0.0008 μL/kg to 8 μL/kg.

In an embodiment, the present invention provides a synergistic herbal composition, wherein the said composition comprises;
(a) cinnamon bark oil in a concentration ranging from 0.33 μL/kg to 22 μL/kg; and
(b) basil oleoresin in a concentration ranging from 0.002 μL/kg to 23 μL/kg.

In an embodiment, the present invention provides a synergistic herbal composition, wherein the said composition comprises;
(a) cassia oil in a concentration ranging from 0.67 μL/kg to 50 μL/kg; and
(b) buchu leaf oil in a concentration ranging from 0.33 μL/kg to 25 μL/kg.

In an embodiment, the present invention provides a synergistic herbal composition, wherein the said composition comprises;
(a) cassia oil in a concentration ranging from 0.67 μL/kg to 50 μL/kg; and
(b) celery seed oil in a concentration ranging from 0.0008 μL/kg to 8 μL/kg.

In an embodiment, the present invention provides a synergistic herbal composition, wherein the said composition comprises;
(a) cassia oil in a concentration ranging from 0.67 μL/kg to 50 μL/kg; and
(b) basil oleoresin in a concentration ranging from 0.002 μL/kg to 23 μL/kg.

In an embodiment, the present invention provides a synergistic herbal composition, wherein the said composition comprises;
(a) buchu leaf oil in a concentration ranging from 0.33 μL/kg to 25 μL/kg; and
(b) celery seed oil in a concentration ranging from 0.0008 μL/kg to 8 μL/kg.

The synergistic herbal composition as claimed in claim 1, wherein the said composition comprises;
(a) buchu leaf oil in a concentration ranging from 0.33 μL/kg to 25 μL/kg; and
(b) basil oleoresin in a concentration ranging from 0.002 μL/kg to 23 μL/kg.

In an embodiment, the present invention provides a synergistic herbal composition, wherein the said composition comprises;
(a) celery seed oil in a concentration ranging from 0.0008 μL/kg to 8 μL/kg; and
(a) basil oleoresin in a concentration ranging from 0.002 μL/kg to 23 μL/kg.

In an embodiment, the present invention provides a synergistic herbal composition, wherein the said composition comprises;
(a) Basil oil in a concentration ranging from 0.027 μL/kg to 57 μL/kg;
(b) Cinnamon bark oil in a concentration ranging from 0.33 μL/kg to 22 μL/kg; and
(c) Cassia oil in a concentration ranging from 0.67 μL/kg to 50 μL/kg.

The aforesaid composition comprising three herbal ingredients extracted from Ocimum basilicum, Agathosma betulina, Cinnamomum cassia, Cinnamomum verum and Apium graveolens is formulated in varying combinations in appropriated concentrations.

In an embodiment, the present invention provides a synergistic herbal composition, wherein the said composition comprises;
(a) Basil oil in a concentration ranging from 0.027 μL/kg to 57 μL/kg;
(b) Cinnamon bark oil in a concentration ranging from 0.33 μL/kg to 22 μL/kg;
(c) Cassia oil in a concentration ranging from 0.67 μL/kg to 50 μL/kg; and
(d) Buchu leaf oil in a concentration ranging from 0.33 μL/kg to 25 μL/kg.

The aforesaid composition comprising the four herbal ingredients extracted from Ocimum basilicum, Agathosma betulina, Cinnamomum cassia, Cinnamomum verum and Apium graveolens is formulated in varying combinations in appropriated concentrations.

In an embodiment, the present invention provides a synergistic herbal composition, wherein the said composition comprises;
(a) Basil oil in a concentration ranging from 0.027 μL/kg to 57 μL/kg;
(b) Cinnamon bark oil in a concentration ranging from 0.33 μL/kg to 22 μL/kg;
(c) Cassia oil in a concentration ranging from 0.67 μL/kg to 50 μL/kg;
(d) Buchu leaf oil. in a concentration ranging from 0.33 μL/kg to 25 μL/kg; and
(e) Celery seed oil in a concentration ranging from 0.0008 μL/kg to 8 μL/kg.

The aforesaid composition comprising the five herbal ingredients extracted from Ocimum basilicum, Agathosma betulina, Cinnamomum cassia, Cinnamomum verum and Apium graveolens is formulated in varying combinations in appropriated concentrations.

In an embodiment, the present invention provides a synergistic herbal composition, wherein the said composition comprises;
(a) Basil oil in a concentration ranging from 0.027 μL/kg to 57 μL/kg;
(b) Cinnamon bark oil in a concentration ranging from 0.33 μL/kg to 22 μL/kg;
(c) Cassia oil in a concentration ranging from 0.67 μL/kg to 50 μL/kg;
(d) Buchu leaf oil. in a concentration ranging from 0.33 μL/kg to 25 μL/kg;
(e) Celery seed oil in a concentration ranging from 0.0008 μL/kg to 8 μL/kg; and
(f) Basil oleoresin in a concentration ranging from 0.002 μL/kg to 23 μL/kg.

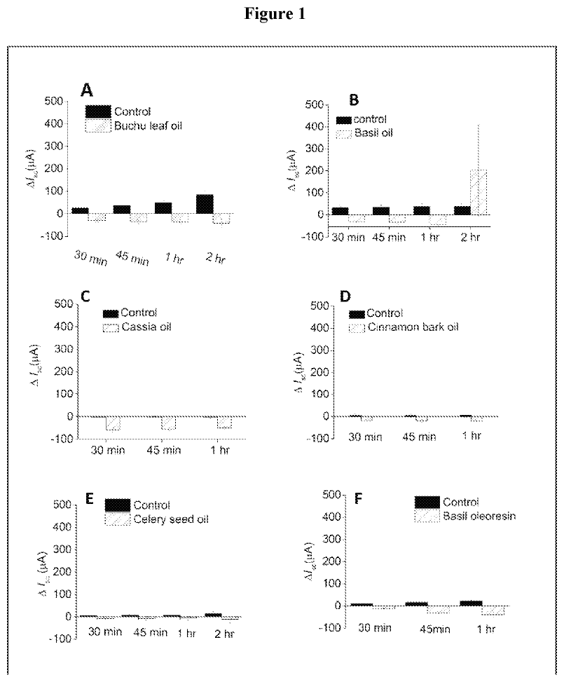

The individual anion secretion inhibitory effect of the components of the herbal composition, including Buchu leaf oil, basil oil, cassia oil, cinnamon bark oil, celery seed oil and basil oleoresin is demonstrated in FIG. 1. Each of the active agents cause decreased anion secretion within a minimum time duration of 30 minutes.

In another preferred embodiment, the present invention provides an herbal composition further comprising electrolytes selected from the group comprising sodium (Na⁺), Cl⁻ (Chloride), HCO₃⁻ (bicarbonate), K⁺ (Potassium), H₂PO₄⁻, HPO₄²⁻ (phosphate ions), Mg²⁺ (Magnesium), Ca²⁺ (calcium) and SO₄²⁻ (sulphate).

Accordingly, the herbal composition comprises one or more of the electrolytes in a concentration ranging from about 8 meq to about 160 meq of Na⁺, about 6 meq to about 156 meq of Cl⁻, about 1 meq to about 45 meq of HCO₃⁻, about 0.5 meq to about 25 meq of K⁺, about 0.1 to about 20 meq of H₂PO₄⁻, about 0.1 meq to about 20 meq of HPO₄²⁻, about 0.1 meq to about 6 meq of Mg²⁺, about 0.1 to 6 meq Ca⁺², about 0.006 to about 6 meq of SO₄²⁻.

More preferably, the present herbal composition comprises 75 meq of Na⁺, 70 meq of Cl⁻, 25 meq of HCO₃⁻, 20 meq of K⁺, 0.4 meq of H₂PO₄⁻, 2.4 meq of HPO₄²⁻, 1.2 meq of Mg′, 1.2 meq Ca′ and 1.3 meq of SO₄²⁻.

In an embodiment, the present invention provides the herbal composition to have pH ranging from 3 to 7.6.

In a further embodiment, the present invention provides the herbal composition having osmolarity ranging from about 50 mOSm to about 325 mOSm.

In another preferred embodiment, the present invention provides a synergistic herbal composition comprising active ingredients selected from the group consisting of oleoresin of Ocimum basilicum, oil from leaves of Agathosma betulina, oil extracted from Cinnamomum cassia, oil extracted from bark of Cinnamomum verum and oil extracted from seeds of Apium graveolens and a pharmaceutically acceptable carrier.

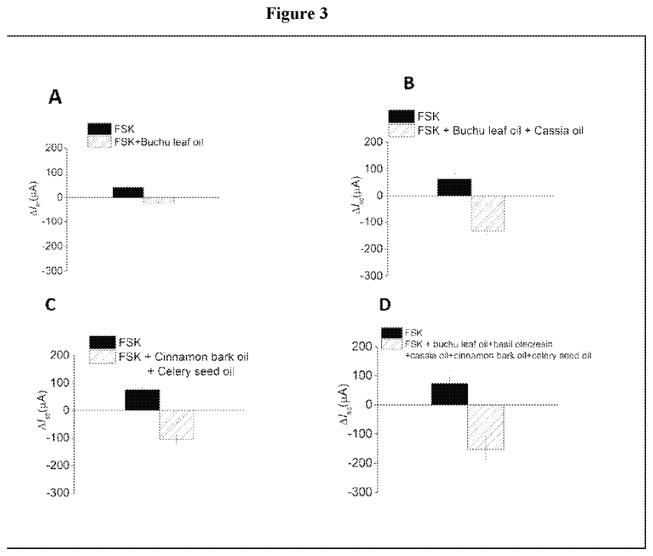

The anion secretion inhibitory activity of the synergistic composition comprising combination of buchu leaf oil, basil oleoresin cassia oil, cinnamon bark oil and celery seed oil against forskolin-stimulated anion secretion is demonstrated in FIG. 3. In an episode of diarrhoea, cAMP activates CFTR channel incorporation and opening of these channels on the brush border membrane of villus epithelial cells, subsequently causing anion secretion. Anion secretion causes electrolyte and fluid loss leading to diarrhoea. The combination of buchu leaf oil, basil oleoresin cassia oil, cinnamon bark oil and celery seed oil resulted in further inhibition of forskolin-stimulated anion secretion (−151.9±43.0 μA/cm² vs 73.5±27.2 μA/cm²) (FIG. 3(D)). Therefore, these results reflect the synergistic anion secretion inhibitory activity of the herbal composition of the present invention against secretagogues such as cAMP, and cGMP involved in the occurrence of diarrhoea.

In another embodiment, the present invention provides an herbal composition having anti-diarrhoeal properties, wherein the said composition is formulated as an oral rehydration solution (ORS), as a powder, syrup, paste, suspension, tablets and intravenous fluid.

Accordingly, the present herbal composition can be administered via the oral or intravenous route.

In yet another embodiment, the present invention provides the herbal composition to further comprise pharmaceutically acceptable additives selected from the group comprising of sweeteners, flavouring agents, masking agents, colorants, preservatives, excipients, gelling agents, oligosaccharides, vitamins, dietary supplements, natural fruit extracts, amino acids and combinations thereof.

In an optional embodiment, the present invention provides the herbal composition comprising glucose.

In another optional embodiment, the present invention provides the herbal composition comprising a zinc source, most preferably elemental zinc.

In yet another preferred embodiment, the present invention provides a method of treating diarrhoea, diarrhoea related disorders and electrolyte and fluid imbalance the said method comprising administering an herbal composition comprising at least one herbal ingredient selected from the group consisting of Basil oleoresin, Buchu leaf oil, Basil oil, Cassia oil, Cinnamon bark oil and Celery seed oil and at least one electrolyte to an individual exhibiting symptoms of diarrhoea.

Accordingly, the present synergistic herbal composition can be administered to correct electrolyte and fluid imbalance, infectious and non-infectious diarrhoea, acute or chronic dehydration, antibiotics-induced diarrhoea, drug-induced diarrhoea, environmental enteropathy, diarrhoea secondary to food allergies, stress-induced diarrhoea, acute shock, hypovolemia, acute severe diarrhoea, hypotension, muscle cramps, Inflammatory Bowel Diseases (IBD) (Crohn's disease, ulcerative colitis), renal diseases, nausea and vomiting.

The present invention provides use of herbal compositions comprising active ingredients selected from the group consisting of oleoresin of Ocimum basilicum, leaf oil of Agathosma betulina, oil extracted from Cinnamomum cassia, oil extracted from bark of Cinnamomum verum and oil extracted from seeds of Apium graveolens and a pharmaceutically acceptable carrier, for treating diarrhoea, diarrhoea related disorders and electrolyte and fluid imbalance.

The following examples, which include preferred embodiments, will serve to illustrate the practice of this invention, it being understood that the particulars shown are by way of example and for purpose of illustrative discussion of preferred embodiments of the invention.

### EXAMPLES

### Example 1: Plant Extracts Screened for Anti-Diarrhoeal Activity

The following active ingredients, plant extracts or compounds were screened initially using the electrophysiological approach and were ranked according to their anti-secretory activity, absorptive capacity or a combination of both, all in the absence of a secretagogue. The active agents/compounds tested include Basil oleoresin, Buchu leaf oil, Basil oil, Cassia oil, Cinnamon bark oil and Celery seed oil.

### Example 2: Extraction Process of Essential Oils and Oleoresins

(a) Extraction Process for Essential Oils
The plant material to be screened for anti-diarrhoeal activity including Ocimum basilicum, Agathosma betulina, Cinnamomum cassia, Cinnamomum verum and Apium graveolen were collected, pre-processed and were steam distilled separately. The water phase was separated and the oil was filtered. The oil was treated with anhydrous 5 mg/10 mL sodium sulphate and was again filtered. Quality control assays were performed and the final product was filtered to obtain the respective oil. The essential oils extracted from each of the plants were stored in sterile containers at room temperature.

(b) Extraction procedure for oleoresins
Ocimum basilicum to be screened for anti-diarrhoeal activity was collected pre-processed and was subjected to solvent extraction. The extract is filtered and desolventised and was blended with additives if required. Quality control assay was performed. The final extract was filtered. The basil oleoresin extracted was stored in sterile containers at room temperature.

### Example 3: Methodology to Assess the Functionality of the Active Ingredients for their Antidiarrheal Properties

Electrophysiological approach was used to identify if the agent under study have unstimulated or basal anti-secretory activity, unstimulated or basal absorptive capacity or a combination of both, all in the absence of secretagogues. Electrophysiological technique will help identifying active secretion and/or absorption in the presence/absence of secretagogues.

### Example 4: Preparation of Formulations

TABLE A

| | | Dose (μl/kg) lower | Higher dose |
|---|---|---|---|
| | Active ingredient | dose studied | studied ml/kg |
| 1 | Basil oil | 1.6 | 3.4 |
| 2 | Cinnamon bark oil | 20 | 1.3 |
| 3 | Cassia oil | 40 | 3 |
| 4 | Buchu leaf oil | 20 | 1.5 |
| 5 | Celery seed oil | 0.05 | 0.5 |
| 6 | Basil oleoresin | 0.14 | 1.4 |

The formulation was first arrived by using 100% of the active agents at their respective K_{M} and functional studies validating the effectiveness of the formulation. Thus, the active agents were used in a 1:1 ratio using 100% of respective K_{M}. However, in separate experiments it was found that different ratios of the active agents in a given formulation could be used to decrease current and therefore increase fluid absorption. Thus, the active agents could be mixed to form a formulation using a range of their different ratios, e.g., buchu leaf oil and cassia oil could be mixed at a ratio of 1:1, 1:2, 1:3, 1:4, 1:5, 1:6, 1:7, 1:8, 1:9 or 1:10. Similarly, the ratios of the two oils could be used in their reverse proportion such that, cassia oil and buchu leaf oil could be mixed at a ratio of 1:1, 1:2, 1:3, 1:4, 1:5, 1:6, 1:7, 1:8, 1:9 or 1:10.

TABLE B
Method for arriving at the concentration of each component used in
the formulation: Active agents were
used in a 1:1 ratio using 100% of respective KM. The ratio
maintained when the agents were used in their
lower and the higher concentration.
The lowest concentration that resulted in a significant
decrease in current was then
used as the lower dose and correspondingly for each of
the active agents. The highest concentration
was similarly derived from the highest concentration of the
substrate that resulted in the maximum reduction in
current in the saturation kinetic analysis.
E.g., basil oil gave a change in chloride secretion at a
concentration of 0.008 μL/5 mL. The lower dose
for kg body weight was therefore
calculated to be 1.6 μL/kg body weight (0.008 * 200 mL).

| | | Cumulative | Cumu- |
|---|---|---|---|
| | | dose studied | lative |
| | Concentration of each | at lowest | dose |
| | component/kg | concentration | studied |
| Combination | body weight | (μL/kg) | at [symbol] |
| 1 + 2 | 1.6 μL/kg, 20 μL/kg | 10.8 | 2.3 |
| 1 + 3 | 1.6 μL/kg, 40 μL/kg | 20.8 | 3.2 |
| 1 + 4 | 1.6 μL/kg, 20 μL/kg | 10.8 | 2.4 |
| 1 + 5 | 1.6 μL/kg, 0.05 μL/kg | 0.82 | 2.0 |
| 1 + 6 | 1.6 μL/kg, 0.14 μL/kg | 0.9 | 2.4 |
| 2 + 3 | 20 μL/kg, 40 μL/kg | 30 | 2.1 |
| 2 + 4 | 20 μL/kg, 20 μL/kg | 20 | 1.4 |
| 2 + 5 | 20 μL/kg, 0.05 μL/kg | 10 | 0.9 |
| 2 + 6 | 20 μL/kg, 0.14 μL/kg | 10 | 1.4 |
| 3 + 4 | 40 μL/kg, 20 μL/kg | 30 | 2.3 |
| 3 + 5 | 40 μL/kg, 0.05 μL/kg | 20 | 1.8 |
| 3 + 6 | 40 μL/kg, 0.14 μL/kg | 20 | 2.2 |
| 4 + 5 | 20 μL/kg, 0.05 μL/kg | 10 | 1 |
| 4 + 6 | 20 μL/kg, 0.14 μL/kg | 10 | 1.5 |
| 5 + 6 | 0.05 μL/kg, 0.14 μL/kg | 0.1 | 1 |
| 1+ 2 + 3 | 1.6 μL/kg, 20 μL/kg, | 20.5 | 2.6 |
| | 40 μL/kg | | |
| 1 + 2 + 3 + 4 | 1.6 μL/kg, 20 μL/kg, | 20.4 | 2.3 |
| | 40 μL/kg, 20 μL/kg | | |
| 1 + 2 + 3 + 4 + 5 | 1.6 μL/kg, 20 μL/kg, | 16.3 | 1.94 |
| | 40 μL/kg, 20 μL/kg | | |
| | 0.05 μL/kg | | |
| 1 + 2 + 3 + 4 + 5 + 6 | 1.6 μL/kg, 20 μL/kg, | 13.6 | 1.9 |
| | 40 μL/kg, 20 μL/kg | | |
| | 0.05 μL/kg, 0.14 μL/kg | | |

The aforesaid values are obtained in studies carried out with mice. To obtain the dosage applicable for human administration, dose conversion from animal to human was performed by multiplying the values by 60 kg. [symbol]
indicates data missing or illegible when filed

The aforesaid formulations (individual oils or their combinations) were prepared and tested in an electrolyte solution containing electrolyte composition as shown below.
75 mM Sodium chloride
20 mM Potassium chloride
1.2 mM Calcium chloride
1.2 mM Magnesium chloride
25 mM Sodium bicarbonate
2.4 mM Potassium hydrogen phosphate
0.4 mM Potassium dihydrogen phosphate

It should be noted that oils can be effective when used alone or in combination in different electrolyte concentrations, a range of total osmolarity (50 mosm to 350 mosm) and range of pH (2.1 to 8.5).

### Example 5: Dose Optimization by Saturation Curve Assay

Saturation kinetics assay was done to determine the km and Vmax of the active ingredients under study. The tissue is treated with increasing concentrations of the active ingredients and is incubated in the Ussing chamber for a particular interval of time. Short circuit current and conductance values were measured after each addition and a curve was plotted with concentration of compounds along X axis and percentage inhibition on the Y axis.

Kₘ and Vₘₐₓ is determined using the classical Michaelis-Menton equation. Kₘ is the concentration of the substrate (isolate), which permits the transporter to achieve half Vₘₐₓ (I_{sc}). The substrate (isolate) exerts its effect on transporters responsible for absorption and/or secretion by activating or inhibiting the transporters. Activation or inhibition of a transporter or channel by a specific substrate (isolate) at a high Kₘ, signifies a low affinity for the substrate, and requires a greater concentration of substrate to achieve Vₘₐₓ. Kₘ for Basil oleoresin, Buchu leaf oil, Basil oil, Cassia oil, Cinnamon bark oil and Celery seed oil determined by saturation curve analysis were 0.016, 0.006, 0.012, 0.008, 0.016 and 0.008 μL/5 mL respectively. The concentrations of the individual oils used in the formulations was calculated from the km (1.75 μl, 2.85 μl, 2.21 μl, 1.0 μl and 0.60 μl for Basil oleoresin, Buchu leaf oil, Basil oil, Cassia oil, Cinnamon bark oil and Celery seed oil respectively). The lowest concentration of the substrate that showed a decrease in short circuit current was used for arriving at the dose.

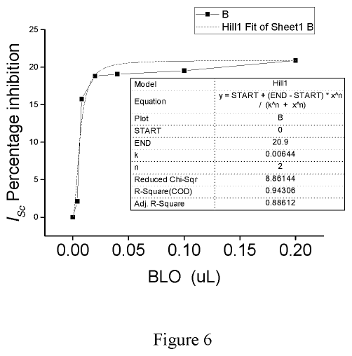

A representative saturation kinetic curve showing the method to arrive at the concentration of buchu leaf oil to inhibit specific channels responsible for electrolyte loss and therefore effective in increasing net fluid absorption is shown in FIG. 6. From the same saturation kinetic analysis, the lowest concentration of buchu leaf oil that was found to reduce the current (Isc) was used to arrive at the lowest concentration of the oil to inhibit the channel responsible for electrolyte loss. Similarly, highest concentration of the given substrate that resulted in maximum reduction in current was used to arrive at the higher dose for the given oil.

The dose range for basil oleoresin is 0.002 μl/kg to 23 μl/kg, buchu leaf oil is 0.33 μl/kg to 25 μl/kg, basil oil is 0.027 μl/kg to 57 μl/kg, cassia oil is 0.67 μl/kg to 50 μl/kg, cinnamon bark oil is 0.33 μl/kg to 22 μl/kg, celery seed oil is 0.0008 μl/kg to 8 μl/kg. Formulations were made using appropriate proportions of the essential oil according to their activity. Saturation kinetics of each of the individual oils that showed significant decrease in anion secretion were conducted to determine the kₘ and Vₘₐₓ. Kₘ was used to determine the optimum concentration of the individual oils in the formulation.

### Example 6: In Vitro Loop Assay: Ileal Sac Assays

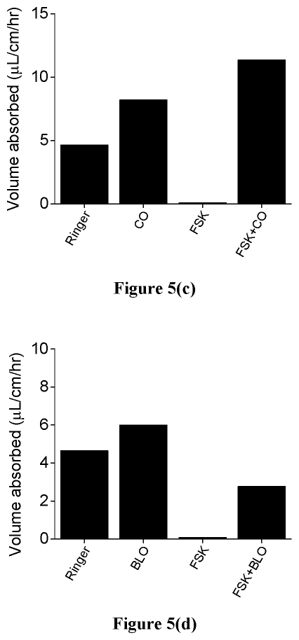

The lowest concentrations of the oils were used for in vitro loop assay. Tissues were exposed to the individual oils at a concentration ranging from 0.001 μL to 5 μL in Ussing chamber. The oils that showed a more potent activity in functional and molecular studies were selected for the loop assay. The lowest concentration that showed a measurable decrease in short circuit current was used for the loop assay (buchu leaf oil 0.1 μL, cassia oil 0.2 μL, basil oil 0.008 μL, cinnamon bark oil 0.1 μL). Combination of oils were used into a formulation using different ratios of the active agents. The formulation was first arrived by using 100% of the active agents at their respective K_{M} and functional studies validating the effectiveness of the formulation. Thus, the active agents were used in a 1:1 ratio using 100% of respective K_{M}. However, in separate experiments it was found that different ratios of the active agents in a given formulation could be used to decrease current and therefore increase fluid absorption. Thus, the active agents could be mixed to form a formulation using a range of their different ratios, e.g., buchu leaf oil and cassia oil could be mixed at a ratio of 1:1, 1:2, 1:3, 1:4, 1:5, 1:6, 1:7, 1:8, 1:9 or 1:10. Similarly, the ratios of the two oils could be used in their reverse proportion such that, cassia oil and buchu leaf oil could be mixed at a ratio of 1:1, 1:2, 1:3, 1:4, 1:5, 1:6, 1:7, 1:8, 1:9 or 1:10. in the same concentration was used for the formulation. Ex vivo studies were performed with ileal segments approximately 10 cm in length that were ligated at one end and then filled with 200 μl of Ringer solution, Ringer solution containing forskolin (10 μM) or Ringer solution containing forskolin plus an agent under study (e.g. buchu leaf oil). The ileal segment was then tied at the other end to form a sac and incubated in Ringer solution bubbled with 95% 02 and 5% CO₂. The beaker was slowly agitated in a water bath at 37° C. After 60-minute incubation, the sacs were removed from the beaker. The volume of the fluid in the ileal sac was measured, and the tissues rapidly cooled. The percentage of net fluid absorption was calculated using the formula (I-O)/I, where I=volume of fluid added to the lumen and O=volume of fluid in the lumen at the end of incubation time (FIG. 5). Mucosa was scraped out from the intestine and brush border membrane was isolated from the tissues. The brush border membrane was used further for western blot analysis.

### Example 7: Functional Assay of Combination of Compounds for their Anti-Secretory Activity in the Presence/Absence of Secretagogues

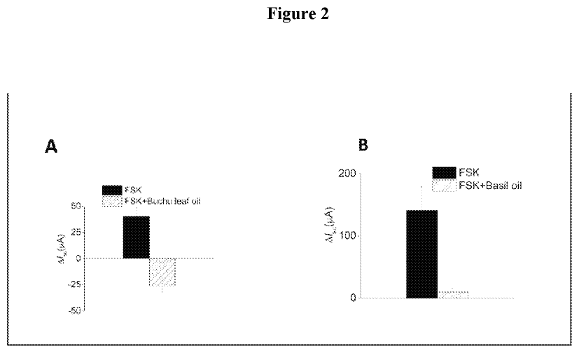

The effect of buchu leaf oil with cassia oil and the combination of buchu leaf oil, basil oleoresin cassia oil, cinnamon bark oil and celery seed oil on forskolin-stimulated anion (Cl⁻ and HCO₃⁻) secretion is given in FIG. 2. It is noted that buchu leaf oil resulted in a significant decrease in forskolin-stimulated anion secretion (−25.7±8.3 μA/cm² vs 40.4±6.7 μA/cm²), but the combination of buchu leaf oil and cassia oil resulted in a far greater inhibition of the forskolin-stimulated chloride secretion (−132.0±12.6 μA/cm² vs 62.3±19.9 μA/cm²). Also, combination of cinnamon bark oil with celery seed oil resulted in significant decrease in forskolin-stimulated anion secretion (−104.1±15.4 μA/cm² vs 75.8±13.0 μA/cm²). Similarly, the combination of buchu leaf oil, basil oleoresin cassia oil, cinnamon bark oil and celery seed oil resulted in further inhibition of forskolin-stimulated anion secretion (−151.9±43.0 μA/cm² vs 73.5±27.2 μA/cm²) (FIG. 3).

### Example 8

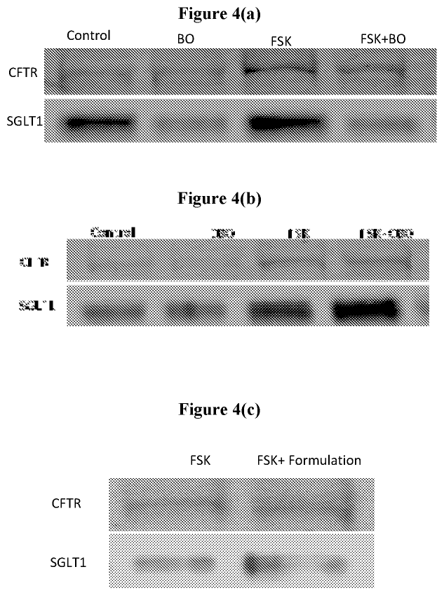

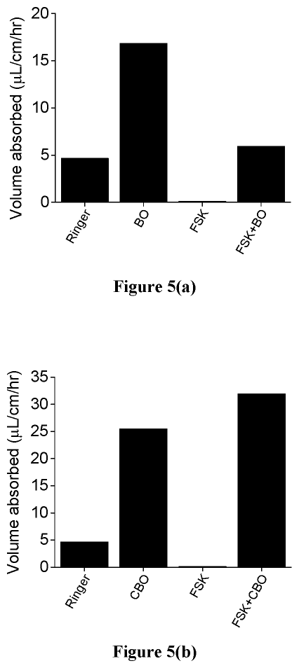

Western blot analysis was done to determine the expression of transporter involved in nutrient dependent and independent electrolyte absorption. Expression of secretory channels CFTR was found to be reduced in presence of the extracts and formulation. Animals at interdigestive phase and digestive phase were used to study the effect of oils on the expression of the transporter SGLT1 during nutrient independent and nutrient dependent phase respectively. Some of the oils increased the expression of SGLT1 and some decreased SGLT1 depends on the phase, whether digestive or inter-digestive (Representative data shown; FIGS. 4(a), (b) and (c)).

### Example 9: The Ussing Chamber System

The present inventors have employed a functional approach for the validation of plant ingredients/compounds for their anti-secretory activity in Ussing chamber. The Ussing chamber provides an in vitro physiological system to measure the transport of ions, nutrients, and drugs across various epithelial tissues. The Ussing chamer system consists of two functional units—The chamber itself and the electric circuitry. Each chamber was filled with the physiological buffer solution, which is continuously aerated and connected with a circulating water bath to maintain the physiological pH and temperature. The electrical circuitry allows the measurement of parameters like short-circuit current, trans-epithelial conductance, voltage, impedence and capacitance. The Ussing chamber system also contains a software support system for data collection (Acquire and Analyze software). Silver/silver chloride voltage and current electrodes are used for the measurement of electrical parameters.

Mice intestinal epithelial tissue was collected by sharp dissection and is opened longitudinally along the entire length. The tissue is cut into 8 segments so that the segments form the entire length of the intestine (from jejunum to illeum) are included in the experiments. The 8 tissues are placed in the 8 channels of the Ussing chamber in a sequential order (jejunum to illeum). The electric circuitry is voltage clamped to zero, to measure the active transport of ions across the membrane. Data is captured using Acquire and Analyze software version 2.3.

### Example 10: Agents that Decrease Active Chloride Secretion

Oleoresins obtained from solvent extraction process and essential oils obtained from steam distillation process were first screened for anti-secretory activity. Oleoresins/oils that showed activity/effectiveness was then ranked based on their anti-secretory activity measured using the electrophysiological approach mentioned. The compounds with the highest unstimulated or basal anti-secretory activity were therefore taken up for further evaluation. Compounds which decreased active chloride secretion/Antidiarrheal agents are enlisted in the table herein below.

Components of the
herbal composition

| | | |
|---|---|---|
| 1 | Oleoresin | Basil oleoresin |
| 2 | Oils | Buchu leaf oil |
| 3 | | Basil oil |
| 4 | | Cassia oil |
| 5 | | Cinnamon bark oil |
| 6 | | Celery seed oil |

The plant extracts were studied for the anti-secretory activity in the presence of a secretagogue such as cAMP. In a diarrhoea episode, increase in intracellular cAMP activates CFTR channel incorporation and opening of these channels on the brush border membrane of villus epithelial cells and subsequently causing anion secretion. Anion secretion causes electrolyte and fluid loss leading to secretory diarrhoea. The extracts screened in the present invention were determined to possess excellent anti-secretory property in the presence of a secretagogues, cAMP induced anion secretion was produced by using forskolin. Representative data of agents that decreased forskolin induced chloride secretion is given in FIG. 2.

### Example 10(A): Synergy of the Present Composition

(i) Fluid Absorption:
The formulation comprising of basil oil, cinnamon bark oil, cassia oil, buchu leaf and celery seed oil at a concentration as described in Table 1, resulted a net fluid absorption of 21 μL/cm/hr when compared to 5.8 μL/cm/hr, 31.8 μL/cm/hr, 11.2 μL/cm/hr, 2.7 μL/cm/hr and 7.6 μL/cm/hr for basil oil, cinnamon bark oil, cassia oil and buchu leaf oil respectively in small intestinal tissues incubated with forskolin.
The formulation comprising of basil oil, cinnamon bark oil, cassia oil, buchu leaf oil, celery seed oil and basil oleoresin at a concentration as described in Table 1, resulted in a net fluid absorption of 11.6 μL/cm/hr when compared to 5.8 μL/cm/hr, 31.8 μL/cm/hr, 11.2 μL/cm/hr, 2.7 μL/cm/hr, 7.6 μL/cm/hr and 8 μL/cm/hr for basil oil, cinnamon bark oil, cassia oil, buchu leaf oil, celery seed oil and basil oleoresin respectively in small intestinal tissues incubated with forskolin.

(ii) Effect of the Present Formulation on Protein Levels of CFTR and SGLT1
For studying synergistic effects, half the dose for each of the compound that showed an effective reduction in forskolin-stimulated increase in short circuit current was used (Data not shown). In order to study the synergy between basil oil and cinnamon bark oil, the said oils were mixed at a concentration of 0.8 ((1.6 μL/kg)/2) and 10 ((20 μL/kg)/2) μL respectively.
When two or more compounds were used together in a formulation, the effect was considered to be synergistic when the desired function was equal or more than what was obtained when used alone or one or more of the compounds neutralized the negative effect it may have in nutrient absorption during digestive or inter-digestive phase (FIGS. 4(a) and 4(b) compared to FIG. 4(c)). Incubating the small intestine with basil oil decreased SGLT1 protein levels suggesting that basil oil decreased glucose stimulated sodium absorption necessitating the importance for including other extracts or agents that will increase SGLT1 protein levels on the brush border membranes when used together as a formulation. Thus the formulation includes extracts or agents that cumulatively increased SGLT1 protein levels on the membrane and their function by demonstrating increased glucose stimulated increase in short circuit current (indicating increased electrolyte and glucose absorption). The reason for including basil oil in the formulation was because, when used together with the formulation, basil oil was found to be most effective in decreasing forskolin stimulated chloride secretion (FIG. 3 D). It also decreased unstimulated and secretagogue induced chloride secretion when used alone (FIGS. 1 B and 2 B). Western blot analysis for SGLT1 showed that forskolin stimulated an increase in SGLT1 expression (FIG. 4 A).

### Example 10(B)

Forskolin stimulated CFTR protein levels decreased in the brush border membrane in mouse intestinal tissues treated with formulation (A combination of 1, 2, 3 and 4) (FIG. 4(c)). These observations agreed very well with electrophysiological observations that the formulation decreased forskolin stimulated increase in current (FIG. 3D). Therefore this formulation can be effectively used for treating secretory diarrhea that is associated with significant electrolyte and fluid loss. In vitro loop assay to measure net fluid movement showed that the formulation increased net fluid absorption when compared to control or basal conditions, indicating decreased electrolyte loss under basal conditions and therefore better hydration. The formulation comprising of basil oil, cinnamon bark oil, cassia oil and buchu leaf oil at a concentration as described in Table 1, resulted in a 4 fold increase in net fluid absorption in small intestinal tissues incubated with forskolin. Also, FSK induced increase in CFTR expression decrease when treated with the formulation, indicating that the formulation can effectively decreased the secretagogue (cAMP) induced chloride secretion. Therefore the formulation could be used in disease conditions that are associated with increased secretagogue-induced electrolyte loss, such as in infectious diarrhea. Western blot analysis for SGLT1 showed that the formulation in the presence of secretagogue further enhanced SGLT1 protein levels on the brush border membrane. Therefore, the formulation can be used to enhance electrolyte and nutrient absorption during diarrheal diseases, associated with increased electrolyte loss.

We claim:

1. A synergistic herbal composition having anti-diarrhoeal properties, comprising herbal ingredients extracted from at least two herbal species selected from the group consisting of Ocimum basilicum (basil), Agathosma betulina, Cinnamomum cassia (cassia), Cinnamomum verum (bark of Cinnamomum) and Apium graveolens (celery).

2. The synergistic herbal composition as claimed in claim 1, wherein the said herbal ingredient is an oleoresin or is an oil extracted from the said herbal species.

3. The synergistic herbal composition as claimed in claim 1, wherein the said oleoresin and essential oils is extracted from aerial or non-aerial parts selected from the group comprising the leaf, stem, roots, bark and seed of the said herbal species.

4. The synergistic herbal composition as claimed in claim 1, wherein the said herbal ingredients is present in a concentration ranging from 50 ml/Kg to 300 ml/Kg.

5. The synergistic herbal composition as claimed in claim 1, wherein the said composition comprises;
(a) basil oil in a concentration ranging from 0.027 μL/kg to 57 μL/kg; and
(b) cinnamon bark oil in a concentration ranging from 0.33 μL/kg to 22 μL/kg.

6. The synergistic herbal composition as claimed in claim 1, wherein the said composition comprises;
(a) basil oil in a concentration ranging from 0.027 μL/kg to 57 μL/kg; and
(b) cassia oil in a concentration ranging from 0.67 μL/kg to 50 μL/kg.

7. The synergistic herbal composition as claimed in claim 1, wherein the said composition comprises;
(a) basil oil in a concentration ranging from 0.027 μL/kg to 57 μL/kg; and
(b) buchu leaf oil in a concentration ranging from 0.33 μL/kg to 25 μL/kg.

8. The synergistic herbal composition as claimed in claim 1, wherein the said composition comprises;
(c) basil oil in a concentration ranging from 0.027 μL/kg to 57 μL/kg; and
(d) celery oil in a concentration ranging from 0.0008 μL/kg to 8 μL/kg.

9. The synergistic herbal composition as claimed in claim 1, wherein the said composition comprises;
(c) basil oil in a concentration ranging from 0.027 μL/kg to 57 μL/kg; and
(d) basil oleoresin in a concentration ranging from 0.002 μL/kg to 23 μL/kg.

10. The synergistic herbal composition as claimed in claim 1, wherein the said composition comprises;
(c) cinnamon bark oil in a concentration ranging from 0.33 μL/kg to 22 μL/kg; and
(d) cassia oil in a concentration ranging from 0.67 μL/kg to 50 μL/kg.

11. The synergistic herbal composition as claimed in claim 1, wherein the said composition comprises;
(c) cinnamon bark oil in a concentration ranging from 0.33 μL/kg to 22 μL/kg; and
(d) buchu leaf oil in a concentration ranging from 0.33 μL/kg to 25 μL/kg.

12. The synergistic herbal composition as claimed in claim 1, wherein the said composition comprises;
(a) cinnamon bark oil in a concentration ranging from 0.33 μL/kg to 22 μL/kg; and
(b) celery seed oil in a concentration ranging from 0.0008 μL/kg to 8 μL/kg.

13. The synergistic herbal composition as claimed in claim 1, wherein the said composition comprises;
(a) cinnamon bark oil in a concentration ranging from 0.33 μL/kg to 22 μL/kg; and
(b) basil oleoresin in a concentration ranging from 0.002 μL/kg to 23 μL/kg.

14. The synergistic herbal composition as claimed in claim 1, wherein the said composition comprises;
(a) cassia oil in a concentration ranging from 0.67 μL/kg to 50 μL/kg; and
(b) buchu leaf oil in a concentration ranging from 0.33 μL/kg to 25 μL/kg.

15. The synergistic herbal composition as claimed in claim 1, wherein the said composition comprises;
(a) cassia oil in a concentration ranging from 0.67 μL/kg to 50 μL/kg; and
(b) celery seed oil in a concentration ranging from 0.0008 μL/kg to 8 μL/kg.

16. The synergistic herbal composition as claimed in claim 1, wherein the said composition comprises;
(a) cassia oil in a concentration ranging from 0.67 μL/kg to 50 μL/kg; and
(b) basil oleoresin in a concentration ranging from 0.002 μL/kg to 23 μL/kg.

17. The synergistic herbal composition as claimed in claim 1, wherein the said composition comprises;
(a) buchu leaf oil in a concentration ranging from 0.33 μL/kg to 25 μL/kg; and
(b) celery seed oil in a concentration ranging from 0.0008 μL/kg to 8 μL/kg.

18. The synergistic herbal composition as claimed in claim 1, wherein the said composition comprises;
(a) buchu leaf oil in a concentration ranging from 0.33 μL/kg to 25 μL/kg; and
(b) basil oleoresin in a concentration ranging from 0.002 μL/kg to 23 μL/kg.

19. The synergistic herbal composition as claimed in claim 1, wherein the said composition comprises;
(a) celery seed oil in a concentration ranging from 0.0008 μL/kg to 8 μL/kg; and
(b) basil oleoresin in a concentration ranging from 0.002 μL/kg to 23 μL/kg.

20. The synergistic herbal composition as claimed in claim 1, wherein the said composition comprises;
(a) Basil oil in a concentration ranging from 0.027 μL/kg to 57 μL/kg;
(b) Cinnamon bark oil in a concentration ranging from 0.33 μL/kg to 22 μL/kg; and
(c) Cassia oil in a concentration ranging from 0.67 μL/kg to 50 μL/kg.

21. The synergistic herbal composition as claimed in claim 1, wherein the said composition comprises;
(a) Basil oil in a concentration ranging from 0.027 μL/kg to 57 μL/kg;
(b) Cinnamon bark oil in a concentration ranging from 0.33 μL/kg to 22 μL/kg;
(c) Cassia oil in a concentration ranging from 0.67 μL/kg to 50 μL/kg; and
(d) Buchu leaf oil in a concentration ranging from 0.33 μL/kg to 25 μL/kg.

22. The synergistic herbal composition as claimed in claim 1, wherein the said composition comprises;
(a) Basil oil in a concentration ranging from 0.027 μL/kg to 57 μL/kg;
(b) Cinnamon bark oil in a concentration ranging from 0.33 μL/kg to 22 μL/kg;
(c) Cassia oil in a concentration ranging from 0.67 μL/kg to 50 μL/kg;
(d) Buchu leaf oil. in a concentration ranging from 0.33 μL/kg to 25 μL/kg; and
(e) Celery seed oil in a concentration ranging from 0.0008 μL/kg to 8 μL/kg.

23. The synergistic herbal composition as claimed in claim 1, wherein the said composition comprises;
(a) Basil oil in a concentration ranging from 0.027 μL/kg to 57 μL/kg;
(b) Cinnamon bark oil in a concentration ranging from 0.33 μL/kg to 22 μL/kg;
(c) Cassia oil in a concentration ranging from 0.67 μL/kg to 50 μL/kg;
(d) Buchu leaf oil. in a concentration ranging from 0.33 μL/kg to 25 μL/kg;
(e) Celery seed oil in a concentration ranging from 0.0008 μL/kg to 8 μL/kg; and
(f) Basil oleoresin in a concentration ranging from 0.002 μL/kg to 23 μL/kg.

24. The synergistic herbal composition as claimed in any one of the preceding claims, wherein the said composition further optionally comprises electrolyte(s) selected from the group comprising sodium (Na⁺), Cl⁻ (Chloride), HCO₃⁻, K⁺ (Potassium), H₂PO₄, HPO₄²⁻, Mg²⁺ (Magnesium), Ca²⁺ (calcium) and SO₄²⁻ (sulphate).

25. The synergistic herbal composition as claimed in claim 24, wherein the said electrolyte(s) is present in a concentration ranging from about 8 meq to about 160 meq of Na⁺, about 5 meq to about 160 meq of Cl⁻, about 1 meq to about 45 meq of HCO₃⁻, about 0.5 meq to about 25 meq of K⁺, about 0.1 to about 20 meq of H₂PO₄⁻, about 0.1 meq to about 20 meq of HPO₄²⁻, about 0.1 meq to about 6 meq of Mg²⁺, about 0.1 to 6 meq Ca⁺², and about 0.006 to about 6 meq of SO₄²⁻.

26. The synergistic herbal composition as claimed in claim 1, wherein the said composition having osmolarity ranging from 50 mOSm to 325 mOSm.

27. The synergistic herbal composition as claimed in claim 1, wherein the said composition is formulated as an oral rehydration solution (ORS), as a powder, syrup, suspension, tablets and intravenous fluid.

28. Use of the herbal composition comprising herbal ingredients extracted from at least one herbal species selected from the group consisting of Ocimum basilicum, Agathosma betulina, Cinnamomum cassia, Cinnamomum verum and Apium graveolens for the treatment of diarrhoea, diarrhoea related disorders and electrolyte and fluid imbalance by exhibiting the following mechanisms;
(i) increasing the non-nutrient and nutrient dependent electrolyte absorption from matured and differentiated villus epithelial cells of the intestine by decreasing active chloride secretion from the crypt of the intestine;
(ii) decreasing the concentration of intracellular secretagogues such as cAMP, cGMP, and Ca²⁺;
(iii) directly inhibiting anion channels, or decreasing the expression of secretory anion channels from the membrane or by increasing the turn-over rate of channels on the brush border membrane;
(iv) increasing electrolyte absorption by increasing the cell turn-over rate; and
(v) increasing electrolyte absorption by increasing the height of villus.

29. Use of the herbal composition comprising herbal ingredients extracted from at least one herbal species selected from the group consisting of Ocimum basilicum, Agathosma betulina, Cinnamomum cassia, Cinnamomum verum and Apium graveolens as a rehydration formulation in infectious and non-infectious diarrhea, Crohn's disease, ulcerative colitis, radiation and chemotherapeutic drug induced gastrointestinal diseases, environmental enteropathy and malnutrition, renal diseases, exercise induced dehydration, dehydrationin old subjects, pre-eclampsia and eclampsia during pregnancy, nausea and vomiting during pregnancy bowel preparation for colonoscopy, nausea and vomiting in pregnancy, astronauts during extended space stay etc.

30. Use of the herbal composition as claimed in claims 28 and 29, wherein the the said composition is prepared in electrolyte(s) selected from the group comprising sodium (Na⁺), Cl⁻ (Chloride), HCO₃⁻, K⁺ (Potassium), H₂PO₄, HPO₄²⁻, Mg²⁺ (Magnesium) Ca²⁺ (Calcium) and SO₄²⁻ (sulphate).

31. Use of the herbal composition as claimed in claims 28 and 29, wherein the concentration of the said herbal composition is ranging from 50 ml/Kg to 300 ml/Kg.

32. A method of treating a subject suffering from diarrhoea, diarrhoea related disorders and electrolyte and fluid imbalance by administering a therapeutically effective concentration of an herbal composition, the said method comprising herbal ingredients extracted from at least one herbal species selected from the group consisting of Ocimum basilicum, Agathosma betulina, Cinnamomum cassia, Cinnamomum verum and Apium graveolens.

33. A method of treating a subject by inducing rehydration in subjects suffering from exercise induced dehydration, dehydration, Crohn's disease, ulcerative colitis, radiation and chemotherapeutic drug induced gastrointestinal diseases by administering a therapeutically effective concentration of an herbal composition, the said method comprising herbal ingredients extracted from at least one herbal species selected from the group consisting of Ocimum basilicum, Agathosma betulina, Cinnamomum cassia, Cinnamomum verum and Apium graveolens.

34. The method of treating as claimed in claims 32 and 33, wherein the said composition is prepared in an electrolyte(s) solution selected from the group comprising sodium (Na⁺), Cl⁻ (Chloride), HCO₃⁻, K⁺ (Potassium), H₂PO₄, HPO₄²⁻, Mg²⁺ (Magnesium), Ca²⁺ (Calcium) and SO₄²⁻ (sulphate).

35. The method of treating a subject as claimed in claims 33 and 34, wherein the said herbal composition is administered in a concentration of the herbal composition is ranging from 50 ml/Kg to 300 ml/Kg.
